(19) United States
(12) Patent Application Publication
(10) Pub. No.: US 2024/0164713 A1
(43) Pub. Date: May 23, 2024

(54) WEARABLE BIOMETRIC SENSORS

(71) Applicant: HID Global Corp., Austin, TX (US)

(72) Inventor: Daniel Henri Raguin, Jupiter, FL (US)

(21) Appl. No.: 18/502,326

(22) Filed: Nov. 6, 2023

Related U.S. Application Data
(60) Provisional application No. 63/383,339, filed on Nov. 11, 2022.

(51) Int. Cl. A61B 5/00; A61B 5/026; A61B 5/11; A61B 5/369; A61B 5/389

(52) U.S. Cl. CPC A61B 5/6803; A61B 5/026; A61B 5/11; A61B 5/369; A61B 5/389

(57) ABSTRACT

A computing device implemented method of detecting compliance in wearing of head-worn Personal Protection Equipment (PPE) is described. The method includes producing one or more biometric sensor signals containing physiological information of a wearer of the PPE using one or more biometric sensors and processing, by the computing device, the physiological information of the one or more biometric sensor signals to detect compliance in wearing of the PPE and generating an indication of compliance in wearing of the PPE.

### PRIORITY APPLICATION

This application claims priority to U.S. Provisional Patent Application Ser. No. 63/383,339, filed on Nov. 11, 2022, the disclosure of which is incorporated by reference herein in its entirety.

### TECHNICAL FIELD

Embodiments illustrated and described herein generally relate to Personal Protection Equipment (PPE) and to techniques to verify compliant use of the PPE.

### BACKGROUND

In a variety of work environments, certain personal protective equipment (PPE) is required. For example, at a construction site, many times hard hats and steel-toed shoes are required to be worn by workers and at a minimum a hard hat for others visiting those areas. Similarly, a machine shop may require safety goggles be used when workers are in certain areas. However, ensuring that individuals wear the required PPE and wear it properly can be a challenge.

### BRIEF DESCRIPTION OF THE DRAWINGS

FIG. 1 is a flow diagram of a computer-implemented method to automatically detect compliance in wearing of head-worn Personal Protection Equipment (PPE).

FIG. 2 is an illustration of an example of PPE that includes biometric sensors.

FIG. 3 is a block diagram of portions of an example of a verifier device.

FIG. 4 is an illustration of a controlled entrance to a controlled area.

FIGS. 5A and 5B are views of a Virtual Reality (VR) headset.

FIG. 6 is a block diagram schematic of portions of another example of a verifier device.

### DETAILED DESCRIPTION

It may be desirable to monitor a work area to ensure that individuals wear PPE in work environments where it is required and to ensure that the PPE is worn properly. A person may be assigned to monitor the work environment for compliance in wearing PPE, but an automated means to verify compliance in use of PPE may be desirable.

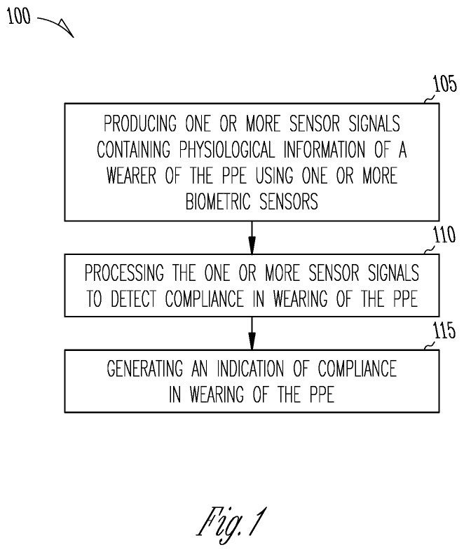

FIG. 1 is a flow diagram of a computer-implemented method 100 to automatically detect compliance in wearing of head-worn PPE (e.g., safety goggles, a hard hat, a helmet, etc.). At block 105, one or more sensor signals are produced using one or more biometric sensors included in the PPE. The sensor signals contain physiological information of a wearer of the PPE. At block 110, the sensor signals are processed by a computing device to detect compliance in wearing of the PPE. At block 115, the computing device generates an indication of compliance (or non-compliance) in wearing of the PPE. This indication may be a signal sent to a separate device indicating that the wearer is compliant or not compliant in wearing the PPE.

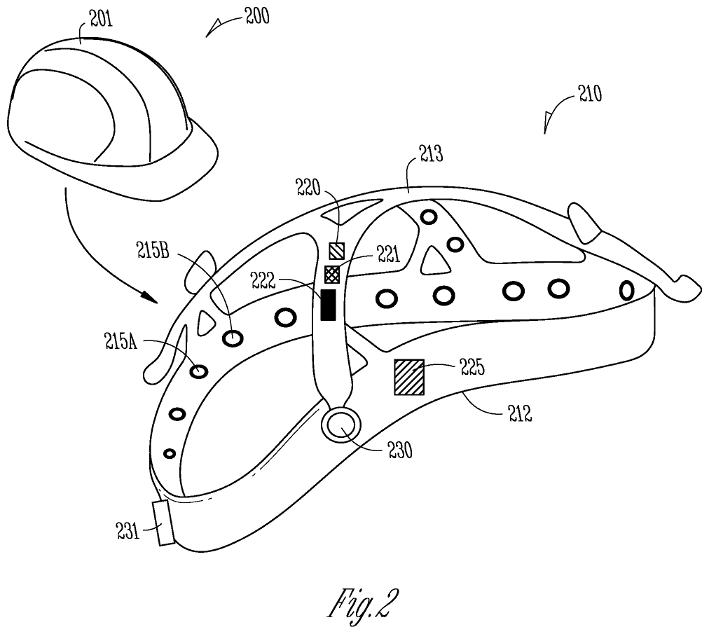

FIG. 2 is an illustration of an example PPE that includes one or more biometric sensors. In the example of FIG. 2, the PPE is a helmet 200, but the techniques described can apply to other types of head-worn PPE as well. The helmet 200 may be a typical non-electronic type of a safety helmet such as a hard hat used by construction workers but may also be one that already has electronics fitted to it such as a helmet used by firefighters that is equipped with thermal cameras and an augmented reality (AR) display. The helmet has a hard outer casing 201 and the inside has a flexible, adjustable support 210 that allows fitting of the helmet to different sized heads. This support system generally comprises a web of straps (for example, left strap 211, right strap 212, and top strap 213) with one or more means 230, 231 of tightening or adjusting the straps to ensure a proper fit.

The helmet 200 includes biometric sensors (e.g., biometric sensors 215a and 215b). The biometric sensors can be integrated into one or more of the straps of the adjustable support 210. The biometric sensors may be attached to the inside of the strap, integrated inside the strap, or mounted on the outside of the strap depending upon the type of sensor and the comfort for the subject. Preferably, the biometric sensors do not stick out from the strap to the extent that they press uncomfortably into the wearer's head. By having the biometric sensors integrated into the straps, the biometric sensors can be in close contact, if not in contact with the wearer's skin. This allows the biometric sensors to make physiological measurements of the wearer in close contact to the wearer such that the signal or signals produced by the biometric sensors can be used to determine if the helmet is worn and if the helmet is properly worn and adjusted by the wearer.

In an example intended to be non-limiting, a biometric sensor can include an electrical signal sensor that senses electroencephalogram (EEG) signal of the wearer or a sensed electromyography (EMG) signal of the wearer. Presence of an EEG or EMG signal, or strength of the EEG or EMG signal may indicate that the helmet is worn properly. Lack of an EEG or EMG signal, or a weak EEG or EMG signal may indicate that the helmet is not being worn or is worn improperly. In another example, a biometric sensor can include a blood flow sensor to provide a blood sensor signal containing blood flow information of the wearer. The blood flow information can in include blood oximetry or heart rate. Presence of blood flow or the strength of the blood sensor signal may indicate that the helmet is worn properly. Lack of blood flow information from the blood sensor may indicate that the helmet is not being worn or is worn improperly. In another example, a biometric sensor can include one or more sensors configured to sense muscle movement (e.g., facial muscle movement) of the wearer. These muscle movement sensors can include one or more of EMG sensors that sense neuromuscular activity as well as pressure sensors or accelerometers that sense physical changes associated with specific muscle movement. Detected muscle activity may indicate that the helmet is worn properly, and lack of any detected muscle movement may indicate that the helmet is not being worn or is worn improperly.

The detected signals may detect normal activity of the wearer of the PPE or head gear or may detect specific gestures or activity by the wearer of this equipment. For example, if the equipment is properly adjusted by the subject and the subject clenches their jaw three times and the sensors and associated processors detect this activity, then the processed signal may be considered as proof the equipment is properly worn or may be used as a signal to activate a certain response in another piece of equipment. Alternatively or in conjunction with detection of specific gestures, the equipment may monitor signals to detect continuous activity, such as normal eye movement for example.

In another example, a biometric sensor can include a video sensor or camera to provide a video signal. For instance, a camera may be attached to the visor area of the helmet. The video signal can be processed by the verifier device to detect eye movement of the wearer or facial muscle movement of the wearer. The eye movement or facial muscle movement can be used to verify that the helmet is worn properly.

Although several biometric sensors are illustrated on the adjustable support 210 in FIG. 2, any suitable number of sensors could be used and could be as few as one. The designer of the helmet 200 may prefer a plurality of biometric sensors for the purposes of measuring more than one physiological parameter of the wearer. Measuring more than one physiological parameter of the wearer may be useful for the purposes of making identification of the wearer easier, for redundancy in case one or more of the biometric sensors fail, and to more precisely ensure that the helmet 200 is properly adjusted (e.g., using biometric sensors on both the left side strap 211 and the right-side strap 212 of support 210 to verify proper adjustment of the helmet 200).

The computing device may include one or more processors to process the sensor signals from the one or more biometric sensors. In FIG. 2, the biometric sensors are connected to processors 220 (e.g., Central Processing Units (CPUs), Application Specific Integrated Circuits (ASICS), Field Programmable Gate Arrays (FPGAs), etc.) containing the appropriate memory, programming (e.g., one or both of software and firmware), and processing power to process the sensor signals completely or partly to detect that the wearing of the PPE is compliant or non-compliant. The helmet 200 can include a transmitter 221 connected to one or more of the processors 220. One or more signals based upon the processed sensor signals may be sent wirelessly to another piece of equipment or a server using transmitter 221. A power source 222 may be incorporated into the helmet if required and preferably is some form of rechargeable battery. For simplicity of the figure, no connections are shown between sensors 215, processors 220, transmitter 221 or power source 222 in the example of FIG. 2, but connections would be present in the PPE as required.

According to some examples, the computing device that processes the sensor signals is separate from the PPE. For example, the biometric sensor or sensors can be included in the PPE and the processors and memory used to process the sensor signals can be included in a separate device that may or may not be worn by the wearer of the PPE. The sensor signals can be sent wirelessly from the biometric sensors to the processor or processors.

In some examples, the sensor signals can be used to identify the wearer. For example, a sensed physiological electrical signal (e.g., an EEG or EMG signal) may include signal features unique to the wearer. The computing device may compare a sensed physiological electrical signal to one or more stored physiological signals to identify the wearer. In another example, a video signal can be used for facial or iris recognition to identify the wearer.

According to some examples, information is transmitted by helmet 200 to a separate device after the biometric sensor signals are fully processed by the electronics of the helmet 200. The signals transmitted by the helmet 200 indicate if the helmet is being worn properly and may include information identifying the person wearing the helmet. The transmitted signal or signals detected from the helmet 200 may also include an identifier (ID) of the helmet to identify the helmet from among multiple helmets present in a controlled area (e.g., a work zone). The transmitted signals can be used to track the location of the helmet.

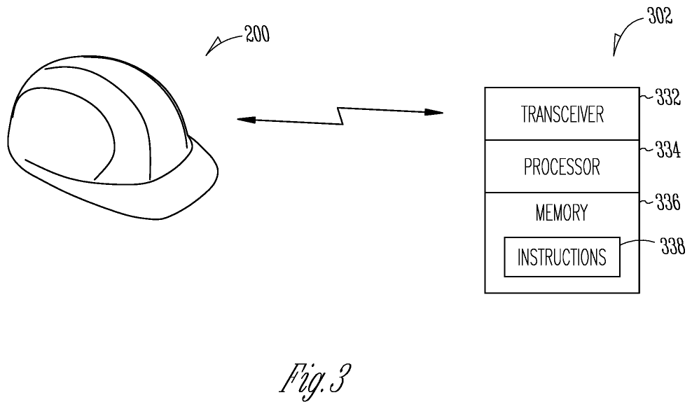

FIG. 3 is a block diagram of portions of an example of a verifier device, which may be, for example, the separate device described above for receiving signals from the PPE electronics. The verifier device 302 includes a receiver or transceiver 332 to wirelessly receive the signals transmitted by the PPE (e.g., helmet 200). The verifier device 302 also includes at least one processor 334 and memory 336. The memory 336 stores instructions 338 that when performed by the processor 334, cause the processor to perform the operations described.

The transmitted signals from the PPE are received by the verifier device 302 and the processor 334 may decode one or both of the ID of the PPE and an indication of compliance in wearing of the PPE included in the received signal. The verifier device 302 can be a server and the verifier device 302 may use the ID of the PPE to track the location of the PPE.

In some examples, the verifier device 302 tracks the location of the PPE in a controlled area. Different techniques can be used to determine the location of the PPE. For example, the circuitry of the PPE may actively generate and transmit a signal in response to a signal sent by the verifier device 302, and the signal from the PPE is used by the verifier device to determine location (e.g., distance from the verifier device). In some examples, the signal used to determine location is a passive signal returned from the PPE back to the verifier device 302 (e.g., a reflected RF or sonar signal).

The verifier device 302 may record the identified PPE being in the controlled area and the indication of compliance in wearing of the PPE in the controlled area. If the received signal includes the ID of the wearer, the verifier device 302 may decode the ID of the wearer in the signal and can record the wearer being in the controlled area and record whether the wearer was compliant in wearing the PPE. In some examples, the verifier device 302 is used to control access to the controlled area.

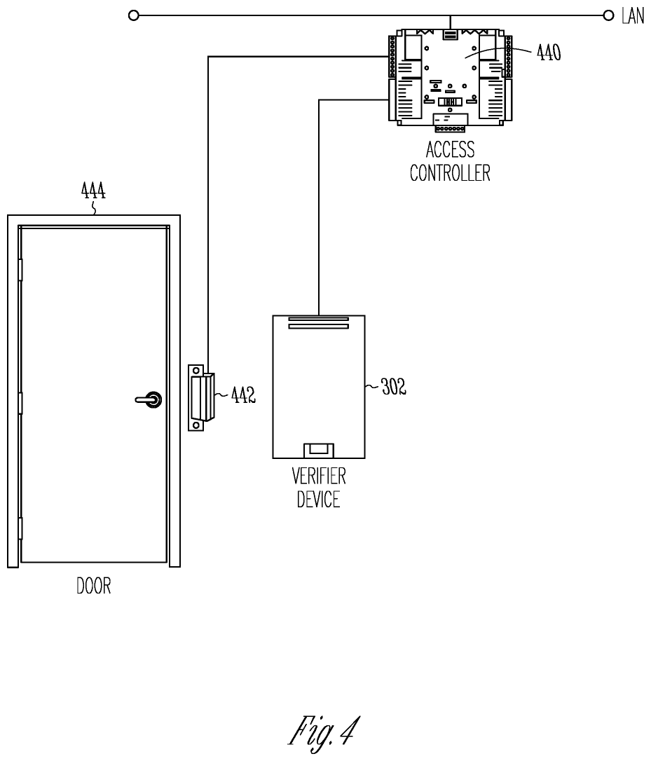

FIG. 4 is an illustration of a controlled entrance (e.g., door 444) to a controlled area. When the wearer approaches the controlled entrance, the PPE sends a signal with the indication of compliance in wearing of the PPE to the verifier device 302. The verifier device 302 may send an enable signal to an access controller 440 that controls an automatic lock 442 on the door 444. The functionality of the access controller 440 may be included in the verifier device 302. If the unlocking mechanism is included in the verifier device 302 as well, the combined device can be referred to as a smart door lock. In some examples, the PPE may transmit a signal to the verifier device 302 that includes the ID of the wearer. The ID of the wearer may have been determined from the biometric sensors as described previously herein. The verifier device 302 may send the ID of the wearer to the access controller 440 that may compare the ID to an access control list to grant or deny access based on the comparison.

The verifier device 302 may be included with a machine or piece of equipment that should be operated while wearing the PPE. The verifier device 302 may be used as a form of interlock for powering on the machine. By way of example, the verifier device 302 may be included with a piece of equipment (e.g., a whisper chipper, lathe, or metal stamper, etc.) and the verifier device 302 may only allow a person to turn on the equipment if the verifier device 302 receives a signal from the PPE indicating one or more of that the PPE is properly worn, that the identity of the person wearing the PPE is one that is allowed to operate that piece of equipment, and that the proximity of the PPE is within a certain distance or in a certain area near the equipment. The proximity of the PPE to the machine may be determined such as by detecting a radio frequency identifier (RFID) signal, near-field communications (NFC) signal, or Bluetooth Low Energy (BLE) communications signal. In the example of FIG. 2, the helmet 200 includes electronics 225 for sending signals for the distance or position sensing by the piece equipment or machine.

If the sensor signals from the biometric sensor or sensors of the PPE are not fully processed by a processor 220, the relevant information may be sent to a verifier device 302 (e.g., a server) wirelessly that fully processes the information to determine one or more of compliance in wearing the PPE, the identity of the wearer, and the ID of the PPE. This information can then be sent back to the PPE which then transmits the information to the piece of equipment.

Alternatively, the verifier device 302 may transmit this information to the one or more pieces of equipment directly using an associated communication system that can be wired or wireless. Preferably, the piece of equipment or machine would still detect signals from the PPE indicating the ID of the PPE and its proximity. The verifier device 302 then determines one or more of that the identified PPE is properly worn, that the identified PPE is worn by an authorized person, or that the PPE is within a certain distance or area to that machine. Based on the determination, the verifier device 302 will release the interlock and allow the piece of equipment to be powered up or maintain the interlock.

According to some examples, the PPE is a helmet for a motorized vehicle such as a motorcycle, all-terrain vehicle (ATV) or race car. The motorcycle helmet can sense that it is properly on top of a person's head and then communicate a signal to a verifier device 302 included in a motorcycle, all-terrain vehicle (ATV), or similar vehicle, that serves as an interlock signal for starting the engine of the motorcycle, ATV, etc. This interlock can also add a level of security for the vehicle. The biometric sensors in the helmet may determine the identity of the wearer of the helmet based upon the biometric sensor signals collected. This information may be used to determine if the person wearing the helmet is authorized to ride the motorcycle, ATV, etc.

The laws requiring helmets to be worn by motorcyclists and ATV riders vary. Helmet laws may vary from country to country, state to state, or municipality to municipality. This motorcycle helmet interlock feature could be tied in with the Global Positioning System (GPS) of the vehicle or GPS of a portable device of the wearer (e.g., a smartphone), so the interlock can be activated in geographic regions where the use of motorcycle helmets is required. In another example, the motorcycle helmet interlock feature may be activated based upon a user setting. For example, regardless of the local or state laws, a parent may want their children to always wear a helmet and hence regardless of GPS position of the motorcycle, ATV, etc., the interlock requiring a helmet to be detected to start the vehicle is always armed. With regards to sensing a passenger, the motorcycle, ATV, etc. may have pressure sensors in the second seat of the vehicle to detect if another person is present. If another person is present, the verifier device 302 may verify that both riders are properly wearing a helmet before allowing the engine of the vehicle to be started or at least engaged out of a neutral engine gear.

In some examples, the PPE can include a head-mounted display. For example, as described previously herein a helmet worn by firefighters can be equipped with thermal cameras and an AR display. AR headsets can be used in a variety of applications where handsfree information is desirable to be projected into a user's field-of-view. Military, fire, police, and medical personal may benefit from the use of AR headsets as they can project thermal or infrared (IR) enhanced scene information, project vital signs of a patient, or information regarding equipment (for example, oxygen content of a breathing apparatus). In another application, repair or maintenance technicians may use AR headsets in order to simultaneously work on a piece of equipment while viewing instructional information pertaining to the task they are currently executing.

A helmet with an AR headset can be outfitted with one or more biometric sensors as described regarding the helmet 200 of FIG. 2. The sensor signals from the biometric sensor or sensors can be used to identify the wearer of the helmet using any of the techniques described herein. The head-mounted display can be connected to one or more processors 220 of the helmet. When the identity of the wearer is determined, the processors 220 can load the wearer's preferences for the head mounted display. Each wearer may have different preferences regarding not only what information should be projected and the location of the information within their field of view on the head-mounted display, but also may have preferences regarding the color, brightness or other attributes of graphics being displayed that are different from those of another wearer.

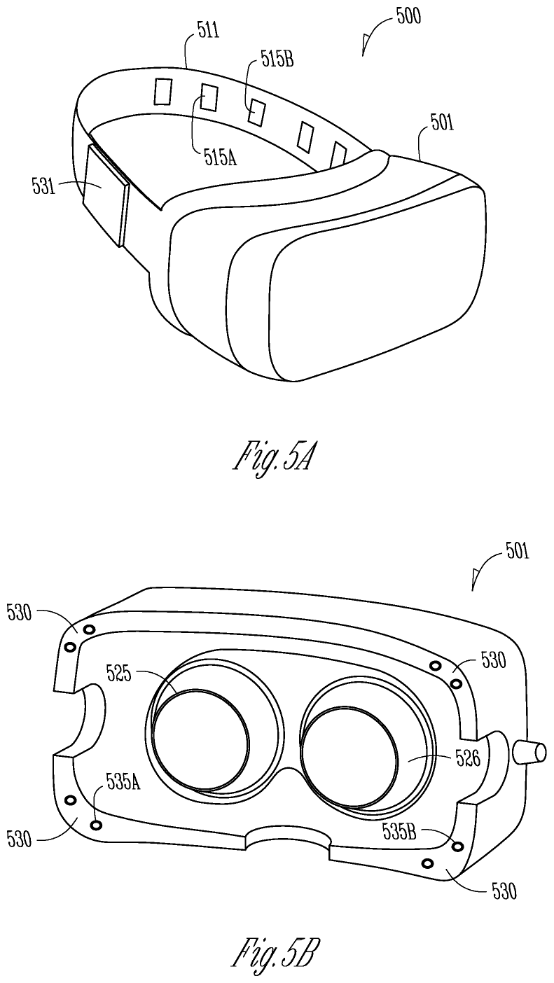

This pre-loading can be applied to virtual reality (VR) displays as well. VR headsets are worn for gaming and simulation/training applications. FIG. 5A shows a VR headset 500 comprised of two basic parts: the front end 501 containing the necessary electronics, optics, and power supply to project the VR display and the strap 511 that allows placement of the VR headset 500 on the head of the user. The strap 511 includes a means of adjustment 531 to fit the VR headset 500 to the head of the wearer.

FIG. 5B is a user view of the front end 501 of the same or different VR headset 500 depicted in FIG. 5A. The front end 501 contains the optics to project the virtual imagery into the user's eyes and electronics to support the optics. In the example of FIG. 5B, separate optical channels 525 and 526 are illustrated for each eye, but the virtual imagery may be projected using a monolithic visor. Optical information is projected onto the monolithic visor that then reflects the optical information into the subject's eyes. The front end 501 may also include head motion tracking sensors to track the field of view of the wearer.

FIG. 5A shows biometric sensors (e.g., 515a and 515b) integrated into the VR headset such as in strap 511. The biometric sensors can be any of the biometric sensors described herein that produce sensor signals. Additionally, FIG. 5B shows biometric sensors (e.g., 535a and 535b) that may be added to a portion or portions 530 of the front end 501 to touch or come close to touching the face of the wearer. Biometric sensors 535a and 535b may be placed and arranged as previously described in relation to the example of FIG. 2 and may be used for one or more of ensuring a correct fit and identification of the wearer.

As described herein regarding FIG. 2, one or more processors, a transmitter, and a power source may be incorporated into the VR headset (not shown in FIGS. 5A and 5B). The one or more processors include a graphics processing unit (GPU) to generate the images to display to the user. The VR headset 500 also includes one or more processors for the processing of the sensor signals and communicating of a processed or partly processed sensor signal to a verifier device 302 wirelessly. The sensor signals may be further processed by the VR headset 500 or a verifier device 302 to identify the wearer of the VR headset 500 and to perform operations in response to the identification, such as to grant authority to the VR headset 500 to run certain programs, resume programs previous started by that user, resume a level of training or gaming for that user, arrange or display the graphic in the VR environment according to the user's preselected preferences, etc. The biometric sensors may also be used to monitor stress or other physiological measures of the wearer's condition and to record these measurements in conjunction with what is occurring in the VR environment or to change the VR environment based upon the wearer's condition.

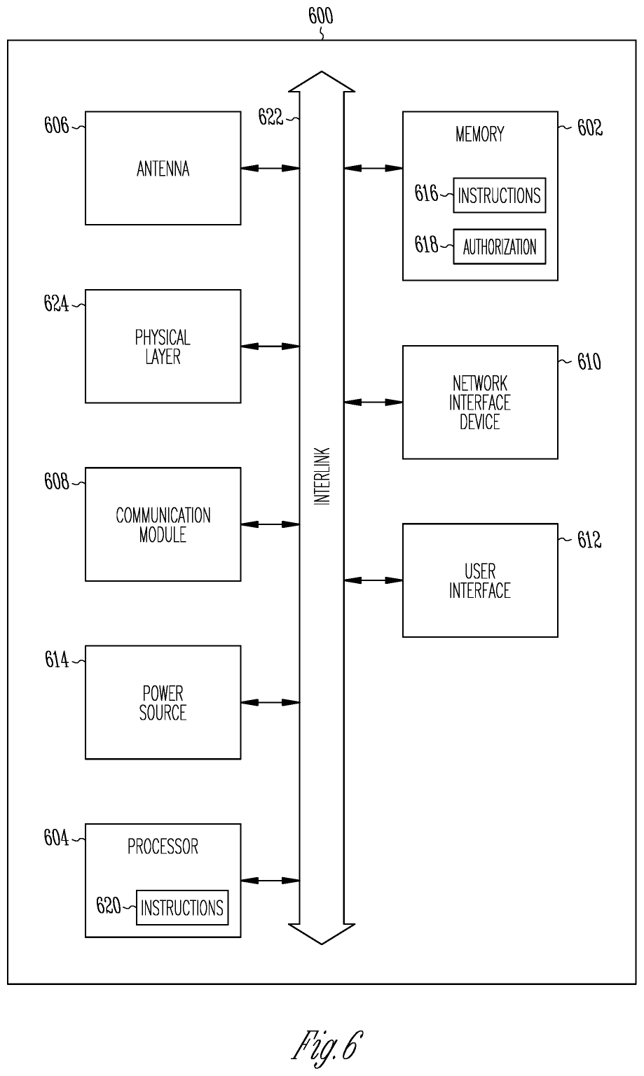

FIG. 6 is a block diagram schematic of various example components of a verifier device of an identity authentication system for supporting the device architectures described and illustrated herein. The device 600 of FIG. 6 could be, for example, a verifier device that receives information regarding proper wear of PPE, or performs processing or partial processing of biometric sensor signals to verify proper wear of PPE. One or more of the components shown in FIG. 6 could be included in the PPE. At a basic level, a verifier device can include an interface (e.g., one or more antennas and Integrated Circuit (IC) chip(s)), which permits the verifier device to exchange data with another device, such as the PPE.

With reference specifically to FIG. 6, additional examples of a device 600 for supporting the device architecture described and illustrated herein may generally include one or more of a memory 602, a processor 604, one or more antennas 606, a communication port or communication module 608, a network interface device 610, a user interface 612, and a power source 614 or power supply.

Memory 602 can be used in connection with the execution of application programming or instructions by processing circuitry, and for the temporary or long-term storage of program instructions or instruction sets 616 and/or authorization data 618, such as identification data, access control data or instructions, as well as any data, data structures, and/or computer-executable instructions needed or desired to support the above-described device architecture. For example, memory 602 can contain executable instructions 616 that are used by a processor 604 of the processing circuitry to run other components of device 600, to make identity determinations based on biometric sensor data, track PPE location, enable equipment based on identity determinations, and/or to perform any of the functions or operations described herein. Memory 602 can comprise a computer readable medium that can be any medium that can contain, store, communicate, or transport data, program code, or instructions for use by or in connection with device 600. The computer readable medium can be, for example but is not limited to, an electronic, magnetic, optical, electromagnetic, infrared, or semiconductor system, apparatus, or device. More specific examples of suitable computer readable medium include, but are not limited to, an electrical connection having one or more wires or a tangible storage medium such as a portable computer diskette, a hard disk, a random access memory (RAM), a read-only memory (ROM), an erasable programmable read-only memory (EPROM or Flash memory), Dynamic RAM (DRAM), any solid-state storage device, in general, a compact disc read-only memory (CD-ROM), or other optical or magnetic storage device. Computer-readable media includes, but is not to be confused with, computer-readable storage medium, which is intended to cover all physical, non-transitory, or similar embodiments of computer-readable media.

Processor 604 can correspond to one or more computer processing devices or resources. For instance, processor 604 can be provided as silicon, as a Field Programmable Gate Array (FPGA), an Application-Specific Integrated Circuit (ASIC), any other type of Integrated Circuit (IC) chip, a collection of IC chips, or the like. As a more specific example, processor 604 can be provided as a microprocessor, Central Processing Unit (CPU), or plurality of microprocessors or CPUs that are configured to execute instructions sets stored in an internal memory 620 and/or memory 602.

Antenna 606 can correspond to one or multiple antennas and can be configured to provide for wireless communications between device 600 and another device. Antenna(s) 606 can be coupled to one or more physical (PHY) layers 624 to operate using one or more wireless communication protocols and operating frequencies including, but not limited to, the IEEE 802.15.1, Bluetooth, Bluetooth Low Energy (BLE), near field communications (NFC), ZigBee, GSM, CDMA, Wi-Fi, RF, UWB, and the like. Antenna(s) 606 may be coupled to one or more physical layers 624 for using any RFID or personal area network (PAN) technologies, such as the IEEE 502.15.1, near field communications (NFC), ZigBee, GSM, CDMA, Wi-Fi, etc.

Device 600 may additionally include a communication module 608 and/or network interface device 610. Communication module 608 can be configured to communicate according to any suitable communications protocol with one or more different systems or devices either remote or local to device 600. Network interface device 610 includes hardware to facilitate communications with other devices over a communication network utilizing any one of a number of transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), mobile telephone networks (e.g., cellular networks), Plain Old Telephone (POTS) networks, wireless data networks (e.g., networks based on the IEEE 802.11 family of standards known as Wi-Fi, or the IEEE 802.16 family of standards known as WiMax), networks based on the IEEE 802.15.4 family of standards, and peer-to-peer (P2P) networks, among others. In some examples, network interface device 610 can include an Ethernet port or other physical jack, a Wi-Fi card, a Network Interface Card (NIC), a cellular interface (e.g., antenna, filters, and associated circuitry), or the like. In some examples, network interface device 610 can include a plurality of antennas to wirelessly communicate using at least one of single-input multiple-output (SIMO), multiple-input multiple-output (MIMO), or multiple-input single-output (MISO) techniques. In some example embodiments, one or more of the antenna 606, communication module 608, and/or network interface device 610 or subcomponents thereof, may be integrated as a single module or device, function or operate as if they were a single module or device, or may comprise of elements that are shared between them.

User interface 612 can include one or more input devices and/or display devices. Examples of suitable user input devices that can be included in user interface 612 include, without limitation, one or more buttons, a keyboard, a mouse, a touch-sensitive surface, a stylus, a camera, a microphone, etc. Examples of suitable user output devices that can be included in user interface 612 include, without limitation, one or more LEDs, an LCD panel, a display screen, a touchscreen, one or more lights, a speaker, etc. It should be appreciated that user interface 612 can also include a combined user input and user output device, such as a touch-sensitive display or the like. Sensor 626 may include a camera, and may be used to capture a biometric, such as a facial image for example.

Power source 614 can be any suitable internal power source, such as a battery, capacitive power source or similar type of charge-storage device, etc., and/or can include one or more power conversion circuits suitable to convert external power into suitable power (e.g., conversion of externally supplied AC power into DC power) for components of the device 600.

Device 600 can also include one or more interlinks or buses 622 operable to transmit communications between the various hardware components of the device. A system bus 622 can be any of several types of commercially available bus structures or bus architectures.

### Additional Disclosure

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. In the above Detailed Description, various features may be grouped together to streamline the disclosure. This should not be interpreted as intending that an unclaimed disclosed feature is essential to any claim. Rather, the subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope should be determined with reference to the appended claims, along with the full scope of equivalents to which such claims are entitled.

What is claimed is:

1. A computing device implemented method of detecting compliance in wearing of head-worn Personal Protection Equipment (PPE), the method comprising:
producing one or more biometric sensor signals containing physiological information of a wearer of the PPE using one or more biometric sensors; and
processing, by the computing device, the physiological information of the one or more biometric sensor signals to detect compliance in wearing of the PPE and generating an indication of compliance in wearing of the PPE.

2. The method of claim 1, including:
sending a signal from the PPE to a device separate from the PPE, wherein the signal indicates the compliance in wearing of the PPE;
detecting, by the separate device, a location of the PPE using the signal;
comparing the detected location of the PPE to a location of a machine; and
enabling, by the separate device, operation of the machine according to the indicated compliance in wearing of the PPE.

3. The method of claim 1, including:
processing the physiological information of the one or more biometric sensor signals to identify the wearer of the PPE;
detecting, using a device separate from the PPE, location of the PPE in a controlled area; and
recording, by the separate device, the identity of the wearer and one or both of wearing of the PPE by the wearer in the controlled area and indicated compliance in wearing of the PPE by the wearer in the controlled area.

4. The method of claim 1, including:
processing the physiological information of the one or more biometric sensor signals to identify the wearer of the PPE;
sending one or more signals from the PPE to a device separate from the PPE that identifies the wearer of the PPE and the location of the PPE;
comparing, by the separate device, the location of the PPE to a machine; and
enabling, by the separate device, operation of the machine according to the detected compliance in wearing of the PPE and the identity of the wearer of the PPE.

5. The method of claim 1, wherein the processing the physiological information of the one or more biometric sensor signals to detect compliance in wearing of the PPE includes:
detecting at least one of eye movement of the wearer or facial muscle movement of the wearer using the one or more biometric sensor signals; and
generating the indication of compliance in the wearing of the PPE according to the detected eye movement or facial muscle movement.

6. The method of claim 1, wherein the processing the physiological information of the one or more biometric sensor signals to detect compliance in wearing of the PPE includes:
detecting blood flow of the wearer using the one or more biometric sensor signals; and
generating the indication of compliance in the wearing of the PPE according to the detected blood flow.

7. The method of claim 1, wherein the processing the physiological information of the one or more sensor signals to detect compliance in wearing of the PPE includes:
sensing one or more physiological electrical signals of the wearer near the temporal area of the wearer; and
generating the indication of compliance in the wearing of the PPE according to the detected physiological electrical signals.

8. The method of claim 9,
wherein the sensing the one or more physiological electrical signals includes sensing at least one of an electroencephalogram (EEG) signal of the wearer or an electromyography (EMG) signal of the wearer; and
generating the indication of compliance in the wearing of the PPE according to the detected one or both of the sensed EEG signal of the wearer or the sensed EMG signal of the wearer.

9. A system comprising:
head-worn personal protection equipment (PPE) including one or more biometric sensors configured to produce one or more biometric sensor signals containing physiological information of a wearer of the PPE; and
a hardware processor and a memory, the memory including instructions that when performed by the processor, cause the processor to process the physiological information of the one or more biometric sensor signals to determine whether the PPE is worn correctly.

10. The system of claim 9, wherein the memory includes instructions that cause the processor to identify a wearer of the PPE using the physiological information of the one or more biometric sensor signals.

11. The system of claim 10,
wherein the PPE includes a head mounted display; and
wherein the memory includes instructions that cause the processor to load preferences of the identified wearer for the head mounted display.

12. The system of claim 10,
wherein the one or more biometric sensors include an electrical signal sensor configured to sense at least one of an electroencephalogram (EEG) signal of the wearer or a sensed electromyography (EMG) signal of the wearer; and
wherein the memory includes instructions that cause the processor to identify the wearer using the at least one of the sensed EEG signal or the sensed EMG signal.

13. The system of claim 10,
wherein the one or more biometric sensors include a video sensor to provide a video signal including image information of the wearer; and
wherein the memory includes instructions that cause the processor to:
detect at least one of eye movement of the wearer or facial muscle movement of the wearer using the video signal; and
generate an indication of compliance in the wearing of the PPE according to the detected eye movement or facial muscle movement.

14. The system of claim 9,
wherein the one or more biometric sensors include a blood flow sensor to provide a blood sensor signal containing blood flow information; and
wherein the memory includes instructions that cause the processor to:
detect blood flow of the wearer using the blood flow sensor signal; and
generate an indication of compliance in the wearing of the PPE according to the detected blood flow.

15. The system of claim 9,
wherein the one or more biometric sensors include an electrical signal sensor configured to sense one or more physiological electrical signals of the wearer near the temporal area of the wearer; and
wherein the memory includes instructions that cause the processor to generate an indication of compliance in the wearing of the PPE according to the detected physiological electrical signals.

16. The system of claim 15,
wherein the electrical signal sensor is configured to sense at least one of an electroencephalogram (EEG) signal of the wearer or an electromyography (EMG) signal of the wearer; and
wherein the memory includes instructions that cause the processor to generate an indication of compliance in the wearing of the PPE according to the at least one of the sensed EEG signal or the sensed EMG signal.

17. The system of claim 9, including a transmitter configured to transmit a wireless signal indicating one or both of an identifier of the PPE and an indication of compliance in wearing of the PPE.

18. The system of claim 9, wherein the PPE is one of safety goggles, a hard hat, or a helmet.

19. A verifier device comprising:
a receiver configured to receive a wireless signal;
at least one hardware processor and a memory, the at least one processor operatively coupled to the receiver and the memory including instructions that when performed by the processor, cause the at least one processor to perform operations including:
decoding an identifier of personal protection equipment (PPE) included in the received wireless signal and an indication of compliance in wearing of the PPE included in the received wireless signal.

20. The verifier device of claim 19, wherein the memory further includes instructions that cause the at least one processor to perform operations including enabling operation of a machine according to the indication of compliance in wearing of the PPE.

21. The verifier device of claim 20, wherein the memory further includes instructions that cause the at least one processor to perform operations including:
detecting location of the PPE relative to a controlled area; and
enabling access to the controlled area according to the indication of compliance in wearing of the PPE.

22. The verifier device of claim 19, wherein the memory further includes instructions that cause the at least one processor to perform operations including:
decoding an identity of the wearer included in the received wireless; and
enabling access to a controlled area according to the identity of the wearer.

23. The verifier device of claim 19, wherein the memory further includes instructions that cause the at least one processor to perform operations including tracking location of the PPE using the wireless signal.

24. The verifier device of claim 20, wherein the memory further includes instructions that cause the at least one processor to perform operations including:
detecting location of the PPE in a controlled area; and
recording the identified PPE being in the controlled area and the indication of compliance in wearing of the PPE in the controlled area.

25. An augmented reality or virtual reality headset, the headset comprising:
a front end portion including optics configured to project virtual imagery to eyes of a wearer of the headset;
one or more biometric sensors configured to produce one or more biometric sensor signals containing physiological information of the wearer; and
processing circuitry configured to produce virtual images to display to the wearer and to process the one or more biometric sensor signals to identify the wearer of the headset.

26. The headset of claim 25, wherein the headset includes a head strap, and the one or more biometric sensors include multiple biometric sensors incorporated into the head strap.

27. The headset of claim 25, wherein the one or more biometric sensors include at least one biometric sensor included in the front end portion.

28. The headset of claim 25,
wherein the one or more biometric sensors include an electrical signal sensor configured to sense one or more physiological electrical signals of the wearer near the temporal area of the wearer; and
wherein the processing circuitry is configured to identify the wearer of the headset using the one or more physiological electrical signals.

29. The headset of claim 25,
wherein the electrical signal sensor is configured to sense at least one of an electroencephalogram (EEG) signal of the wearer or an electromyography (EMG) signal of the wearer; and
wherein the processing circuitry is configured to identify the wearer of the headset using the at least one of the sensed EEG signal or the sensed EMG signal.

30. The headset of claim 25, wherein the processing circuitry is configured to load preselected display preferences according to the identity of the wearer of the headset.

31. The headset of claim 25, wherein the processing circuitry is configured to grant access to a virtual reality soft program according to the identity of the wearer of the headset.

32. The headset of claim 25, wherein the processing circuitry is configured to resume playing of a previously loaded virtual reality software program according to the identity of the wearer of the headset.

33. The headset of claim 25, wherein the processing circuitry is configured to record physiological information of the wearer included in the one or more the biometric sensor signals.
